(19) United States
(12) Patent Application Publication
(10) Pub. No.: US 2019/0376110 A1
(43) Pub. Date: Dec. 12, 2019

(54) METHOD TO DETECT BACTERIAL ACTIVITY AND PATHOGENICITY WITH BIODYNAMIC SENTINELS

(71) Applicant: Purdue Research Foundation, West Lafayette, IN (US)

(72) Inventors: Michael R. Ladisch, West Lafayette, IN (US); David D. Nolte, West Lafayette, IN (US); John J. Turek, West Lafayette, IN (US); Eduardo de Aquino Ximenes, West Lafayette, IN (US); Honggu Choi, West Lafayette, IN (US)

(73) Assignee: Purdue Research Foundation, West Lafayette, IN (US)

(21) Appl. No.: 16/434,224

(22) Filed: Jun. 7, 2019

Related U.S. Application Data
(60) Provisional application No. 62/682,191, filed on Jun. 8, 2018.

(51) Int. Cl. C12Q 1/18; G01N 21/31

(52) U.S. Cl. CPC C12Q 1/18; G01N 33/04; G01N 21/31

(57) ABSTRACT

Biodynamic imaging (BDI) performs Doppler spectroscopy of intracellular motion in living samples. The present disclosure describes novel methods and systems to perform: 1) BDI of living 3D tissue culture exposed to bacteria; 2) BDI of living biopsies exposed to bacteria; 3) BDI of infected tissues responding to antibiotics. A novel new element is the use of immortalized cancer cells to generate tissues that act as “biosensors” or “reporters” of bacterial infection, for cells as found directly in aqueous samples and cells that have been concentrated through filtration, centrifugation or a combination while maintaining them in a viable form. Pathogenicity is assessed through the spectral Doppler signatures of the changes in tissue dynamics induced by the bacteria.

### CROSS-REFERENCE TO RELATED APPLICATION

This application claims the benefits of U.S. Provisional Application Ser. No. 62/682,191, filed Jun. 8, 2018, the contents of which are incorporated herein entirely.

### TECHNICAL FIELD

The present disclosure relates to novel methods and systems for biodynamic imaging of bacterial activity and pathogenicity by using intracellular Doppler spectroscopy. Pathogenicity is assessed through the spectral Doppler signatures of the changes in tissue dynamics induced by the bacteria.

### BACKGROUND

This section introduces aspects that may help facilitate a better understanding of the disclosure. Accordingly, these statements are to be read in this light and are not to be understood as admissions about what is or is not prior art.

Finding biological pathogens requires recovery, concentration and interrogation of small numbers of pathogenic bacteria in environmental samples containing large numbers of non-pathogenic bacteria and other debris. Current technologies face practical limitations, particularly in rapidly determining phenotypes of small numbers of isolated microorganisms, most of which are not pathogenic. Conventional polymerase chain reaction (PCR) and genomic technologies for pathogen detection require that cell numbers be increased to detectable levels, i.e., enriched using microbiological methods. Enrichment requires time and limits the speed and accuracy of interrogation, introducing the possibility of false negatives, and does not readily determine unknown pathogens.

Therefore, there is a need to develop methods and detecting systems for rapidly detecting of the presence of unknown pathogens.

### SUMMARY

The present disclosure relates to novel methods and systems for biodynamic imaging of bacterial activity and pathogenicity by using intracellular Doppler spectroscopy. Pathogenicity is assessed through the spectral Doppler signatures of the changes in tissue dynamics induced by the bacteria.

In one embodiment, the present disclosure provides a method for detection of pathogens in viable microorganisms, wherein the method comprises a) interacting living immortalized cells with viable microorganisms, and b) measuring changes in Doppler spectra.

### BRIEF DESCRIPTION OF THE DRAWINGS

FIG. 1 shows the biodynamic imaging (BDI) system configuration. BDI uses a Mach-Zehnder interferometer with a short coherence (˜20 μm) light source. There are two optical paths which are the object and reference arm, respectively. A sample is placed in the object arm and incident light illuminates the biological specimen.

FIG. 2 shows examples of optical coherence images (OCI) and motility contrast images (MCI).

FIG. 3a to FIG. 3f show the back-scatter brightness and motility (NSD) for E. coli, Salmonella and Listeria pellets as a function of time responding to application of nutrient and negative controls. The three curves are for depleted media, brine and fresh nutrient, respectively.

FIG. 4 shoes baseline spectra for E. coli, Salmonella and Listeria. The spectra in all three samples are featureless with 1/f character.

FIG. 5 shows the example of a Doppler-edge fit to the bacterial pellet response to a nutrient shock. The average edge frequency and Doppler number are shown for two times: one immediately after the nutrient application, and another 2 minutes later, showing a rapid relaxation back towards the 1/f spectrum.

FIG. 6 shows the response of E. coli, Salmonella and Listeria pellets to nutrient. The dashed curves are the baseline before the dose. The response spectra were taken 2 minutes after the dose.

FIG. 7 shows the rapid differential response of E. coli, Salmonella and Listeria pellets to a nutrient shock. These responses occurred within 2 minutes of the application of nutrient to the wells.

FIG. 8 shows the nutrient shock response on pellets of e. coli, Salmonella and Listeria, respectively. Dramatic low-frequency enhancement occurred (the Doppler edge). The nutrient shock showed the strongest response in e. coli (10 times). Spectra were normalized by the 3^{rd} baseline spectrum.

FIG. 9 shows the biodynamic spectrogram response of E. coli pellet to ethanol (70%). The response is inhibitory with strong 60% suppression in the mid and lower frequencies that represents inhibited motion at the lowest speeds.

FIG. 10 shows the spectrograms of e. coli after applying NaOH and bleach respectively. Low frequency is highly suppressed after adding chemical agents.

FIG. 11 shows the response of E. coli, Salmonella and Listeria pellets to Cipro. The dashed curves are the baseline before the dose. The response spectra were taken 2 minutes after the dose.

FIG. 12 shows the differential spectral response of pellets within 2 minutes to Ciproflaxicin for the three bacterial species of this study. There is very little change at the high frequencies between 1 Hz and 10 Hz. The low-frequency response is rapid and decays rapidly. Listeria has the weakest response to Cipro.

FIG. 13 shows Cipro response spectrograms of E. coli, Salmonella and Listeria pellets.

FIG. 14 shows the range of sizes of intracellular components versus characteristic Doppler frequency. The lowest frequencies relate to cell shape changes that can be associated with cell death. The mid frequencies relate to membrane undulations and nuclear motion that can be associated with cell division, membrane transport and replication processes. The highest frequencies relate to organelle and vesicle transport that can be associated with metabolism and cellular energy production.

FIG. 15a shows NSD for E. coli and Salmonella infecting DLD spheroids with two negative controls (no bacteria and Listeria innocua). The replicate numbers range from 3 to 15. FIG. 15b shows Normalized back scattering brightness changes over time.

FIG. 16 shows the initial and final OCI images of DLD-1 spheroids infected by ecoli (top row) and Listeria monocytogenes (bottom row). There is considerable brightening in both cases.

FIG. 17 shows the physical refractive-index contrast mechanism for using light scattering to detect bacteria that adhere to the surface of cells (e.g., E. coli) or internalize (e.g., Listeria monocytogenes).

FIG. 18 shows the spectrum of DLD-1 under infection by 10⁷ #/mL of E. coli. The time per loop is 2 minutes.

FIG. 19 shows tissue dynamics spectrograms of DLD-1 tissue spheroids infected by a) E. coli, b) Salmonella enterica, c) Listeria innocua and d) Listeria monocytogenes.

FIG. 20 shows the time traces of selected spectral ranges for the rheology band (10 mHz) and the organelle transport band (2 Hz-6 Hz) for an applied exposure of 10⁷ cfu per ml.

FIG. 21 shows examples of bacterial infection of biodynamic sentinels by Listeria and Salmonella compared to sentinel that were treated one hour after infection. The antibiotic prevents the infection from being established in the sentinel.

FIG. 22 shows the effect of penicillin G on Salmonella infection of DLD-1.

### DETAILED DESCRIPTION

For the purposes of promoting an understanding of the principles of the present disclosure, reference will now be made to the embodiments. It will nevertheless be understood that no limitation of the scope of this disclosure is thereby intended.

In the present disclosure the term “about” can allow for a degree of variability in a value or range, for example, within 10%, within 5%, or within 1% of a stated value or of a stated limit of a range.

In the present disclosure the term “substantially” can allow for a degree of variability in a value or range, for example, within 90%, within 95%, or within 99% of a stated value or of a stated limit of a range.

The present disclosure relates to novel methods and systems for biodynamic imaging of bacterial activity and pathogenicity and sensitivity to antibiotics by using intracellular Doppler spectroscopy.

The present disclosure describes novel methods and systems to perform: 1) BDI of living 3D tissue culture exposed to bacteria; 2) BDI of living biopsies exposed to bacteria; 3) BDI of infected tissues responding to antibiotics. A novel new element is the use of immortalized cancer cells to generate tissues that act as “biosensors” or “reporters” of bacterial infection, for cells as found directly in aqueous samples and cells that have been concentrated through filtration, centrifugation or a combination while maintaining them in a viable form. Pathogenicity is assessed through the spectral Doppler signatures of the changes in tissue dynamics induced by the bacteria.

Biodynamic imaging is a three-dimensional deep-tissue optical imaging technique that uses intracellular motion as an image contrast to image living samples in vitro. It performs laser ranging up to a millimeter deep inside highly scattering tissue or other media. As the light scatters from moving constituents of the living sample, the light is Doppler shifted in frequency. Multiple simultaneous biological processes produce a superposition of Doppler beats that is collected as dynamic speckle. Fluctuation spectroscopy of the time-varying speckle intensity produces Doppler spectra of the living sample. Different frequency ranges relate to different scales of intracellular processes, broadly separating out different internal processes that are affected by treatments or by drugs with different mechanisms of action. The shifts of the Doppler spectral content in response to the treatment represent drug-response fingerprints that measure how the living target is affected by the treatment.

In the context of cancer research, the response of cancer tissues to chemotherapy applied in vitro and measured with biodynamic imaging provides a method for predicting the response of patients to their chemotherapy. A preclinical laboratory trial using biodynamic imaging on ovarian cancer xenografts in mice predicted sensitivity to platinum therapy with 100% accuracy. A preclinical trail using biodynamic imaging on canine patients with B-cell lymphoma predicted sensitivity to CHOP therapy with 84% accuracy. Pilot trials on human breast cancer and esophageal tissues responding to neoadjuvant chemotherapy based on doxorubicin are currently under way and displaying similar accuracies.

The present disclosure provides novel methods and detecting systems than can rapidly detect the presence of unknown pathogens using a tissue fragment of an immortal cell line as a sensitive detector named here a sentinel. This method is better than conventional methods because it is more sensitive, rapid, and is able to detect unknown pathogenic bacteria. A change in intracellular motion of the sentinel cause by the bacteria infection acts as a reporter of the presence of the bacteria. The method can also be used to detect food pathogens such as Listeria and Salmonella.

This disclosure describes a new research direction using biodynamic imaging to interrogate bacterial targets. Prior art consists of low-coherence imaging, biodynamic imaging, and fluctuation spectroscopy. There are several features that render this new invention non-obvious in light of previous art. One is the formation of bacterial pellets that have adhesion properties and light scattering properties that maximize the interaction of light with bacterial motions. It was not a priori obvious that bacterial pellets would have cohesive properties appropriate for BDI. It was also not a priori obvious that light scattering would be sensitive to Doppler shifts in bacterial pellets. Light scattering is well-known prior art and has been used to monitor free bacteria (see Vargas, S., B. E. Millan-Chiu, S. M. Arvizu-Medrano, A. M. Loske and R. Rodriguez. “Dynamic light scattering: A fast and reliable method to analyze bacterial growth during the lag phase.” Journal of Microbiological Methods 137: 34-39. (2017), but the high density and opaque nature of the bacterial pellets used in this disclosure would lead one skilled in the art to conclude that dynamic light scattering would not be viable. Another feature is the use of the vast range of immortalized cell lines to grow living tissues that behave as a “canary in a coal mine” or as a sentinel, otherwise known as a biosensor, to measure effects of bacterial infection. This invention shows how to match the cell type to the bacteria (for instance esophageal tissue is relevant for digestive tract infection). One more feature is identifying bacteria through their internallization behavior, with adhered bacteria displaying different signatures than internalized bacteria.

The methods and systems useful in the processing of food samples to detect pathogenic cells in the samples can be found in U.S. Pat. No. 9,651,551.

In one embodiment, the present disclosure provides a method for concentrating pathogens and detecting their sensitivity to antibiotic treatments, wherein the method comprises a) a concentration of the pathogens into pellets, and b) the exposure of the pellet to antibiotic agents while measuring Doppler spectra. The system to be used is given in U.S. Pat. No. 9,651,551, the method is unique to the current invention.

In another embodiment, the present disclosure provides a method for detection of pathogens in viable microorganisms, wherein the method comprises a) interacting living immortalized cells with viable microorganisms, and b) measuring changes in Doppler spectra.

In one aspect, the method for detection of pathogens comprises detecting expressed metabolites from said viable microorganisms by measurements based on Doppler shift. In one aspect, the method comprises detecting pathogens through expressed metabolites from said viable microorganisms by measurements based on Doppler shift.

In one aspect regarding the method for detection of pathogens, wherein said viable microorganisms is obtained by a combination of microfiltration to concentrate viable microorganisms extracted from a food produce to achieve sufficient (1 to 10,000) numbers of living bacteria needed to achieve detection of the presence of said microorganisms.

In one aspect regarding the method for detection of pathogens, wherein the pathogens are represented in a pellet. In one aspect, the pellet comprises between 100 and 10,000 viable microorganisms. In one aspect, he pellets comprise toxins associated with living (viable) or dead bacterial cells.

In one aspect regarding the method for detection of pathogens, said viable microorganisms are living and/or dead bacteria. In one aspect, the microorganisms may be either gram positive or gram negative bacteria.

In one aspect regarding the method for detection of pathogens, said viable microorganisms comprises living and nonpathogenic bacteria.

In one aspect regarding the method for detection of pathogens, wherein said viable microorganisms are obtained by a combination of microfiltration and/or ultrafiltration to concentrate viable microorganisms to achieve sufficient number of living bacteria needed to achieve detection of the presence of said microorganisms, wherein the number of the living bacteria is in the range of 1-1,000,000, 1-100,000, 1-10,000, or 1-1000.

In one aspect regarding the method for detection of pathogens, wherein the viable microorganisms are concentrated and recovered from aqueous extracts of biological materials selected from the group consisting of food produce, fruit, meats, and dairy products.

In one aspect regarding the method for detection of pathogens, wherein expressed metabolites are obtained from viable microorganisms extracted from a food produce to achieve toxin concentrations needed to achieve detection as biomarkers associated with pathogens within 8 hours.

In one aspect, the method for detection of pathogens is achieved within 8 hours.

In one aspect regarding the method for detection of pathogens, the method comprises using living immortalized cells to detect presence of monocultures of viable microorganisms pathogens.

In one aspect regarding the method for detection of pathogens, said pathogens are detected by using a living tissue fragment of the living immortalized cells as a sensitive detector.

In one aspect regarding the method for detection of pathogens, the living immortalized cells are living immortalized cancer cells. In one aspect, the cancer cells are colon cancer cells.

In one aspect, expressed metabolites and toxins are obtained from viable microorganisms extracted from a food produce to achieve toxin and pathogen concentrations needed to achieve detection as biomarkers associated with pathogens within 8 hours where toxins are concentrated by membranes having a lower molecular cutoffs (from 1 kD to 200 kD) and viable pathogens and the toxins associated with them re concentrated using microfiltration membranes with cut-offs of 200 kD or greater. Bacterial cells are concentrated with microfiltration membranes having size cutoffs of 0.45 micrometer to 2 micrometer.

In one aspect, the method for detection of pathogens comprises using living immortalized cells to detect presence of monocultures of viable microorganisms pathogens.

In one embodiment, the present disclosure provides a method for concentrating microorganisms and detecting environmental perturbations on the microorganisms wherein the method comprises a) a selective concentration of microorganisms and formation of concentrated pellets, and b) detection of environmental perturbations on the microorganisms using light scattering Doppler spectroscopy.

In one embodiment, pathogens and/or their associated toxins of the viable pathogens are processed by employing hollow fiber filtration in order to concentrate bacterial cells in a living (viable) form suitable for interrogation using living immortalized cells.

Experimental Configuration

Sample Manipulation

Various bacterial specimens can be prepared in a pellet form. A pellet is a condensed form of bacteria which allows them to be carried and handled more efficiently. Bacteria are grown in LB medium for 4 to 12 hours. When culturing is done, the bacteria density in medium is approximately 10⁸ #/mL. To make the pellet, 25 mL of bacteria medium is stored in a centrifuge tube. The medium is centrifuged by 10,000 rpm for 10 minutes (Avanti J-20 XPI). During centrifugation, a pellet is formed at the bottom of a centrifuge tube.

Pellets of bacteria from environmental samples of biological materials (produce, fruits, meats, dairy products, are recovered by physical concentration using microfiltration) followed by centrifugation. The microfiltration step captures the viable microorganisms in a reduced volume of buffer or water, so that the volume is small enough to contain a concentrated forms of living bacteria that may centrifuged to form a pellet. This overcomes the difficulty of achieving the same result if he same number of viable bacteria are found in a larger volume of fluid of 100 mL or greater, where centrifugation to form a pellet is hindered by the geometry of the centrifuge bottles that are required to hold the larger volume of cell suspension, and the time needed to process the more dilute form of the bacterial cells into a pellet.

For an infection assay, there is a wide range of possible immortal cell types known collectively as “cell lines” that are derived from many different organs or tissues. Many of these can be grown from a few cells into a tissue fragment that can be used as sentinels to monitor bacterial infection.

As one example of an infection assay, a DLD-1 colon cancer cell line is prepared as a tissue fragment sentinel. DLD-1 cell lines are cultured for 24 hours and form a spheroid shape after incubation. Spheroids are immobilized within two hours by poly-D-lysine after placing spheroids on a bottom of plate.

To obtain infection spectra, bacteria in a pellet form can be re-suspended by diluting a pellet with various media (5% NaCl, growth medium, PBS etc.). The diluting process produces a uniform concentration of bacteria, and the bacterial concentration can be controlled by controlling volumes of media.

Plate Setup

There are two different sample plate preparations. A monoplex plate is a plate preparation format to test a rapid response of bacteria in single wells. The time resolution of the spectrum measurement is 2 minutes. A multiplex plate is a mode for measuring slow behavior using a measuring period is 30 minutes. For the multiplex, 16 wells are measured in parallel. A plate is firmly fixed on a translational stage controlled at 37 degrees Celsius or other temperature depending on the cell type and the bacterium. The plate is covered with a semi-permeable membrane which prevents the medium from drying out while permitting air molecule exchange.

Optical Configuration

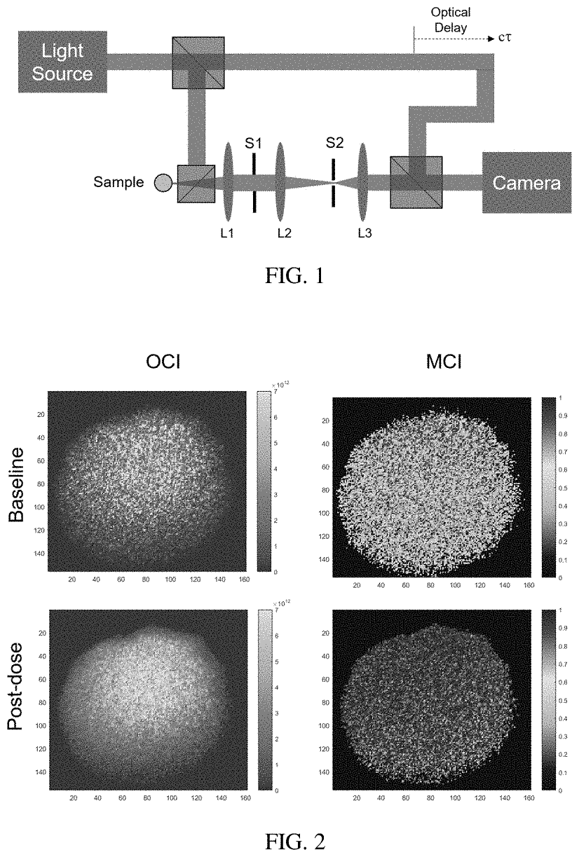

The optical configuration of the BDI system is shown in FIG. 1. Backscattered light from a specimen forms a speckle field consisting of many speckles with sizes determined by the point-spread-function of the imaging optics, and the speckle is delivered by a 4-f system (L1 and L2, f=15 cm). After the 4-f system, a lens (L3, f=5 cm) conducts Fourier transformation of the speckle and the Fourier image is formed at a Fourier plane on a Fourier-domain pixel array (such as a CCD). Simultaneously, an optical delay in the reference arm is adjusted to selectively interfere the Fourier signal at the Fourier plane with the purpose to select light from a selected depth inside the tissue (or pellet) target. The interference occurs only if the optical paths of the reference and the object arm are identical, therefore the coherence-gate technique selectively interferes signals coming from a fixed depth inside the sample.

Frequency Bands

Bacteria move through water with maximum speeds up to 30 microns per second, but because of the run-and-tumble dynamics, there is a continuous velocity spectrum. Biodynamic imaging currently measures the frequency window from approximately 0.01 Hz to approximately 10 Hz, corresponding to speeds from about 3 nanometers per second to about 3 microns per second, which are well within the range of the continuous speed distribution. Higher frame-rate cameras can extend this range to 30 microns per second.

Biodynamic Imaging of Bacterial Pellets

Bacterial pellets are a unique target that are well suited for the capabilities of biodynamic imaging. Biodynamic imaging is capable of imaging through dense scattering media, using short-coherence gating to optically section the target. The highly motile nature of bacteria presents another advantage for biodynamic imaging that uses cellular motion as its image contrast.

OCI and MCI

Examples of optical coherence images (OCI) and motility contrast images (MCI) are shown FIG. 2. More specifically, FIG. 2 shows the baseline and postdose images of an E. coli bacterial pellet for OCI and MCI. The speckle character changes dramatically in the OCI image after adding nutrient. The NSD also increases dramatically (Baseline NSD=0.63, post-dose NSD=0.92). The OCI format is an optical section that is color coded on scattering intensity. The optical section is approximately 20 microns thick, located 100 to 200 microns above the bottom of the plate. The baseline OCI image is acquired prior to the application of nutrient (Lysogeny broth, 1% tryptone, 0.5% bacto yeast extract, 1% sodium chloride, 0.1% glucose and 1.5% bacto agar), and the post-dose is 2 minutes after adding nutrient. The MCI format is the temporal normalized standard deviation (NSD) of the same optical section. The post-dose activity is highly activated for this case of E. coli responding to a nutrient shock. For Poisson statistics, the highest achievable NSD is unity, although other statistical processes can produce NSD values slightly larger than unity.

Backscatter Brightness and Motility

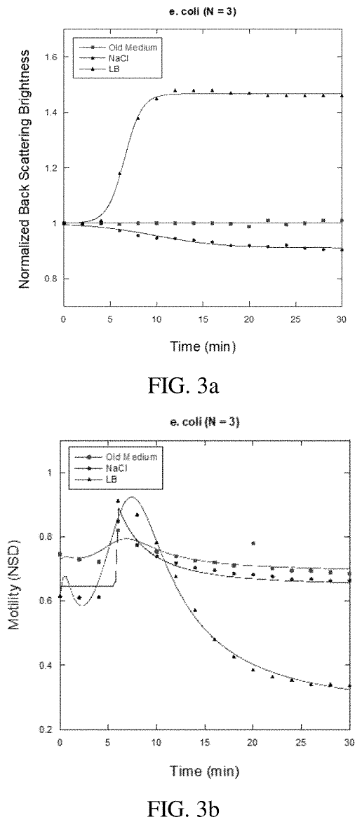

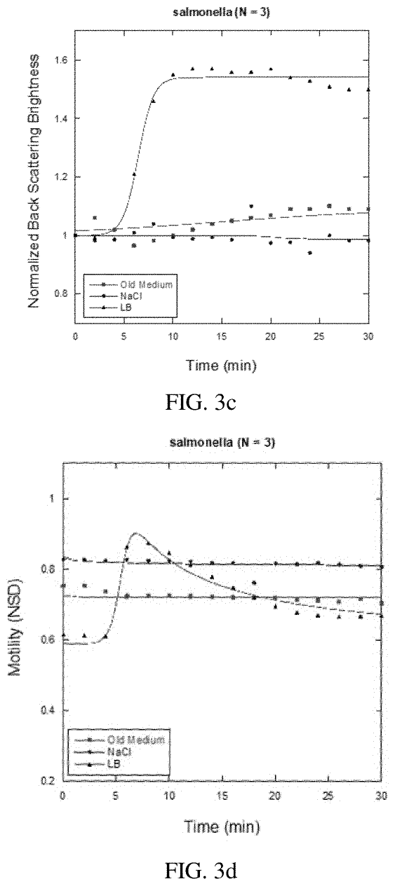

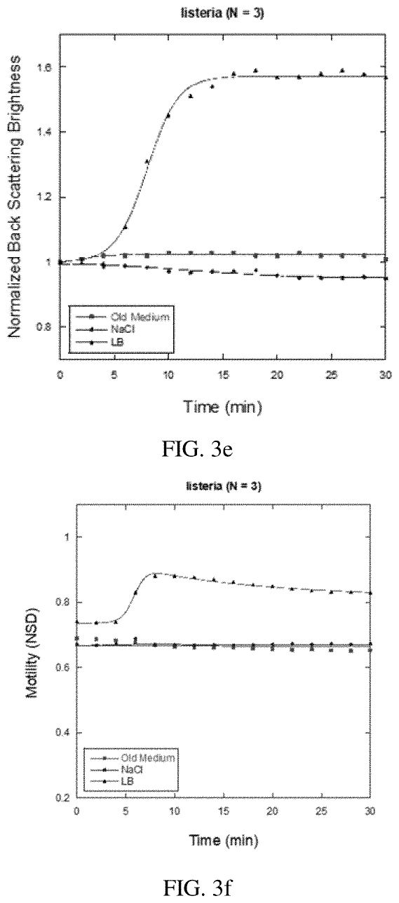

The loose cellular packing of the bacterial pellets is easily disrupted by vigorous shaking. In addition, this loose assemblage is also susceptible to environmental shocks, as from sudden exposure to nutrients or to antibiotics. The bacteria in steady state are relatively quiescent (in terms of motility), but can be activated to a high motility state by the perturbation. The integrated back-scatter brightness and the NSD are shown in FIG. 3a-FIG. 3f for E. coli, Salmonella and Listeria. The three curves are for depleted media, brine and fresh nutrient media (LB), respectively. In all cases there is a dramatic increase in motility for LB media accompanied by large increase in integrated back-scatter brightness, while the depleted media and the brine show no significant change. The integrated backscatter brightness increase saturates to a new value within about 6 minutes and is stable. The motility shows a nearly instantaneous increase followed by a slower decrease that takes place over about 10 minutes. The motility eventually stabilizes at a lower value than the starting value for E. coli, but remains elevated in the case of Listeria and Salmonella.

An increase in backscatter brightness is caused by increased optical heterogeneity in the pellet. The fact that it stabilizes at a higher value than the starting value in all three bacteria cases suggests that there is a permanent (over at least 20 minutes) change in the physical property of the pellet, or in the physical property of the individual bacterial cells, caused by the nutrient. The very sudden increase in the motility is certainly caused by the nutrient shock, but the later decrease could in part be caused by an increased backscatter brightness (because the NSD is the standard deviation of the intensity divided by the brightness).

Fluctuation Spectroscopy

To gain a more specific understanding of the effect of the nutrient shock, fluctuation spectroscopy is used in a mode called fluctuation spectroscopy. This breaks down the effects of a perturbation into a range of frequencies that are altered by the perturbation. Fluctuation spectroscopy is used routinely in biodynamic imaging applications for drug assays on cancer tissue. In the context of the bacterial pellets, the perturbation similarly can cause spectral changes whose specific frequencies relate to specific speeds of biological processes involved.

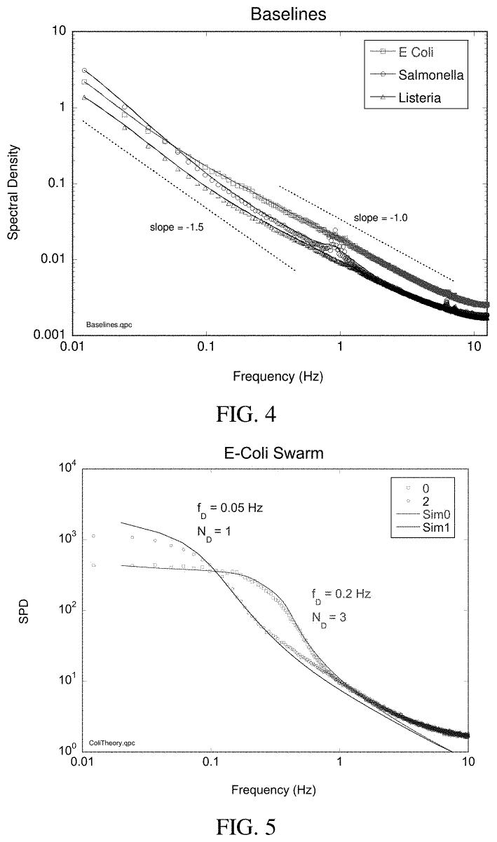

The baseline spectra for E. coli, Salmonella and Listeria are shown in FIG. 4. The frequency span is from 0.01 Hz to 10 Hz. For the backscatter geometry at 840 nm wavelength infrared this span corresponds to rms speeds of about 3 nanometers/second to about 3 microns/second. The key aspect of the baseline spectra of the bacterial pellets is the lack of any spectral feature in the spectra. The spectra are characterized generally as 1/f spectra which is common for many noise processes that contain a hierarchy of scales. The wide range of bacterial biological processes in the quiescent pellet would contribute to this 1/f character.

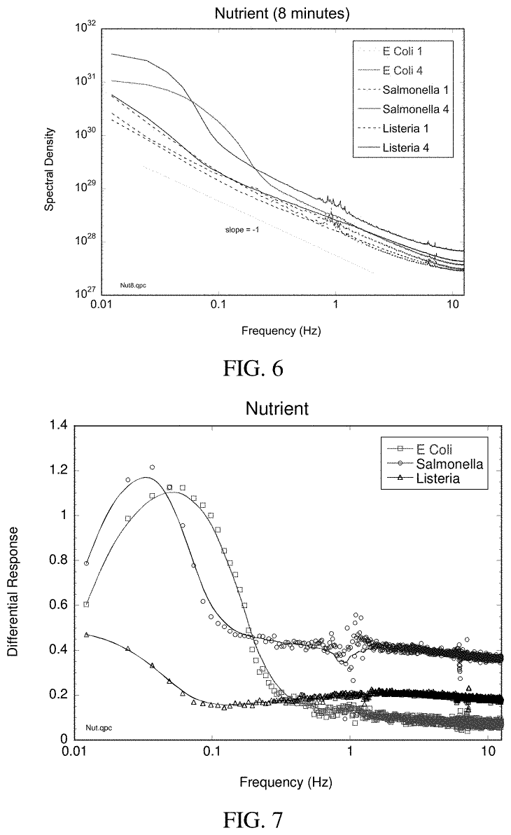

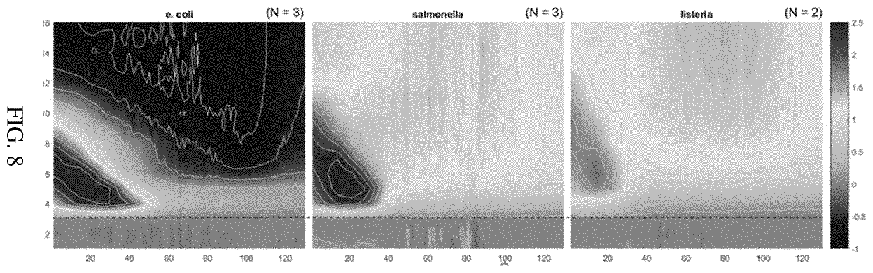

The response of an E. coli pellet to a nutrient shock is shown in FIG. 5. Immediately after the application of the nutrient, a distinct feature called a Doppler edge emerges from the spectrum. A Doppler edge is a spectral feature that is caused by a relatively uniform speed within the biological sample. The isotropic averaging of the Doppler shift over all orientations converts a linear-transport Doppler peak into a Doppler edge. The fast-response Doppler edge frequency for E. coli is 0.2 Hz (corresponding to speeds of about 100 nm/second). The Doppler number that fits this feature is equal to N_{D}=3. This Doppler number fit is a lower bound on the microscopic Doppler numbers which would have a spread of speeds. Within 2 minutes the Doppler edge has already relaxed to 0.05 Hz with a lower-bound Doppler number of 1. This rapid relaxation of the enhanced motion in response to the nutrient shock is consistent with the rapid change in NSD values in FIG. 3. It should be noted that the swarm speed of 100 nm/second is much slower than the free swimming speed of E. coli that is as large as 30 microns per second. The packing of the bacteria are likely to significantly inhibit the swimming speed of the bacteria. The responses of all three bacterial species to nutrient are shown in FIG. 6. E coli displayed the strongest response, followed by Salmonella. The response of Listeria to nutrient is relatively weak. The differential response is shown in FIG. 7, and the corresponding spectrograms are shown in FIG. 8. Both Salmonella and Listeria show enhanced broad-frequency behavior in response to the nutrient, while for E. coli it has a broad-frequency inhibition at long times.

Fluctuation Spectroscopy of Antimicrobial Agents

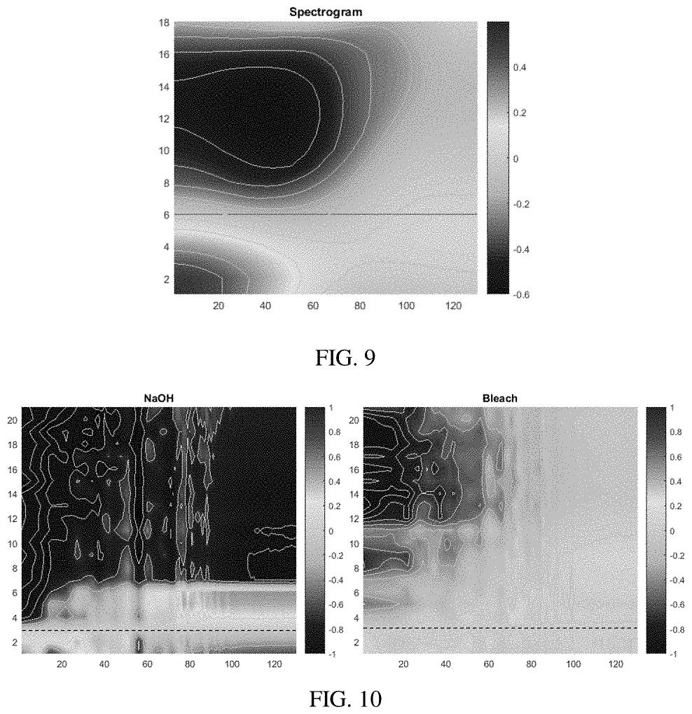

A key opportunity for biodynamic imaging of bacterial activity is the ability to monitor the efficacy and mechanisms of antimicrobial agents. It will be increasingly important to identify antibiotic resistance phenotypes as more strains of bacteria become resistant. It is projected that by 2050 more people will die of resistant bacterial infections than cancer. An example of the inhibition of an E. coli pellet by 70% ethanol is shown in FIG. 9. The mid and low frequencies are inhibited by approximately 60%, while the highest frequency is relatively unaffected. The response of E. coli to NaOH and bleach are shown in FIG. 10. The responses of ethanol and bleach are similar, while NaOH shows broad inhibition of activity (broad-frequency cell death).

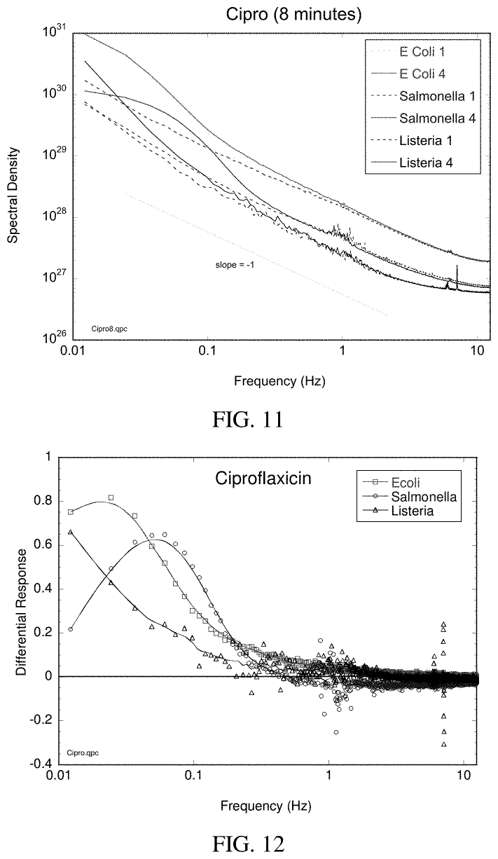

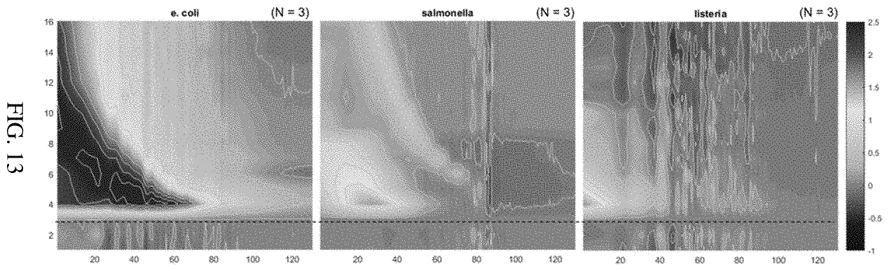

The responses of E. coli, Salmonella and Listeria pellets to the antibiotic Ciprofloxacin (Cipro) are shown in FIG. 11, the differential change in frequency is shown in FIG. 12, and the corresponding spectrograms are shown in FIG. 13. It is striking that the response to Cipro is very similar to the response to nutrient. This may be because these are all the short-time responses (total response within 30 minutes of dose), and Cipro may affect the bacteria metabolically in this short time frame in a manner similar to nutrient. It will be extremely important to extend the time-frame of these experiments to span several doubling times to investigate whether the similar responses of nutrient and Cipro deviate at longer times. Listeria has the weakest response to Cipro. The highest frequencies in all three cases are not affected. This is in contrast to the Nyquist floor in the case of the nutrient shock where the activity at the Nyquist floor increases after exposure to the fresh nutrient.

In conclusion, bacterial pellets are extremely sensitive to environmental disturbances. The resting baseline spectra for all three bacterial species had featureless 1/f character. Exposure to a sudden change in fresh nutrient or antibiotic Cipro produces a fast and pronounced Doppler edges that rapidly (over several minutes) relaxes back to the baseline spectrum. The response to Cipro relative to nutrient has small differences that may require longer monitoring times (several doublings), and there are minor differences in the changes induced at the Nyquist floor.

Bacterial Infection of Living Tissue Sentinels

Tissue Sentinels

Small living tissue fragments consisting from about 30 cells to a million cells represent a special kind of biosensor, here called a biodynamic sentinel. They function in close analogy to a “canary in a coal mine”. The living motion inside living tissue changes its motions when infected by bacteria. The dynamic motion inside living tissue has a broad Doppler spectrum with different frequency bands associated with different intracellular mechanisms, each that can be affected in a different way by invading bacteria.

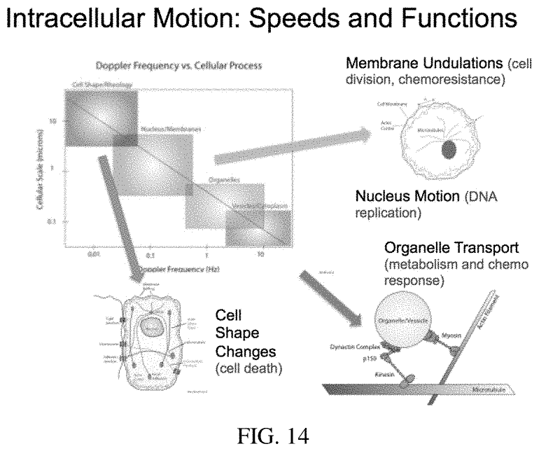

The different frequency ranges are shown in FIG. 14 that relates the size of the intracellular components to their characteristic Doppler frequency. The lowest frequencies relate to cell shape changes that can be associated with cell death such as apoptosis or necrosis. The mid frequencies relate to active membrane undulations and nuclear motion that can be associated with cell division, membrane transport and replication processes. In chemotherapy testing, the mid frequency has strong correlation with response of a patient to treatment. The highest frequencies relate to organelle and vesicle transport that can be associated with metabolism and cellular energy production. Active processes inside the cells are detected with high specificity by measuring the Nyquist floor at the high-frequency limit of the spectrum defined as half of the frame rate.

The biodynamic sentinels have a vastly larger degree of flexibility than a canary in a coal mine because of the vast number of different types of immortalized cell lines they can be grown from. Because bacteria have evolved to invade specific niches related to different tissue types, the sentinels can be grown from a range of tissue types to match the expected identity of the target bacteria. For instance, many bacteria infect the gastro-intestinal track, and there are many immortalized cell lines that are relevant, such as esophageal, stomach, intestine, colon and bowl. Sentinels could be grown from any or all of these cell lines and used to monitor bacterial infection specific to a certain cell type.

Effect of Infection on Intracellular Motility

Immortalized cancer cell lines are a common and versatile resource as three-dimensional tissue surrogates for the purpose of studying cellular processes in microenvironments that simulate natural living tissues. These tissue constructs have characteristic biodynamic spectra that span three orders of magnitude across three decades of Doppler frequencies and tend to have characteristic frequencies, known as knee frequencies that are a single broad spectral feature. Different cell lines can display different knee frequencies depending on how cohesive the tissue is.

Bacterial infection of living tissue occurs through several different mechanisms. For instance Listeria monocytogenes actively punctures the cell membrane, while Salmonella coopts the actin processes of the cell to allow them to take up the bacterium. E coli, in contrast, passively attaches to the exterior of the cell. These different mechanisms may be expected to alter the underlying cellular dynamics of the tissue in different ways that may be related back to the pathogenicity of the bacteria.

As an example, fast motion of Listeria or Salmonella moving through the cytosol at speeds of about 1 micron per second produce frequency contributions at the high frequency range of about 1 Hz to 10 Hz. Increasing bacterial load or activity would display on a spectrum as an increased Nyquist floor. Alternatively, extracellular crowding caused by proliferation of E. coli dampens membrane motions which would display on a fluctuation spectrum as a suppressed mid-frequency on the spectrum. As the sentinel cells begin to die because of the infection, necrosis would cause blegging of the cell membrane which is detected as an increased spectral content at the lowest frequencies.

Biodynamic Imaging of Tissue Sentinel Infection

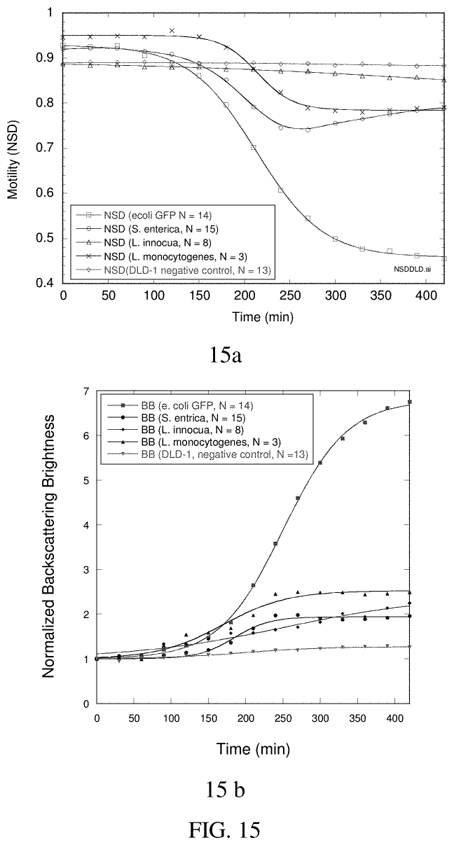

An example of biodynamic data from bacteria infection of a three-dimensional tissue grown from the DLD-1 adenocarcinoma cell line is shown in FIG. 15, monitoring the motility and the integrated back-scatter brightness in response to the infection. Two negative controls are the medium without bacteria, and medium infused with the Listeria serotype Listeria innocua that is nonpathogenic. The negative controls had little to no influence on the underlying cellular dynamics of the tissue.

The three strains of active bacteria were E. coli, Listeria monocytogenes and Salmonella enterica—the last two of which are pathogenic. The concentration of bacteria in these cases were 10⁷ #/mL. The motility was altered in all three cases, and the intracellular motility was inhibited by the bacterial infection, except for Listeria monocytogenes that showed an initial short-term increase. In the three bacterial cases, there is an initial period of approximately 150 minutes during which little change in intracellular motility occurs, followed by a relatively sudden suppression that takes place over approximately 100 minutes as the motility is suppressed to a lower steady value. Surprisingly, the largest impact on intracellular motility was by the non-pathogenic E. coli infection, with smaller changes induced by the pathogenic species. It is possible that the E. coli bacterial load is very high, altering the medium conditions by depleting nutrient, altering pH and expelling metabolites that are mildly toxic. In the case of L. mono and Salmonella, it is possible that the growing bacterial load is smaller than for E. coli. It is also possible that the suppression in intracellular motility of the tissue is partially offset by an increased motility attributed to the bacterial activity inside the cytoplasm. For instance, in the case of L. mono, the toxic effects of the bacterial load may be offset by high cytoplasmic activity of the replicating bacteria.

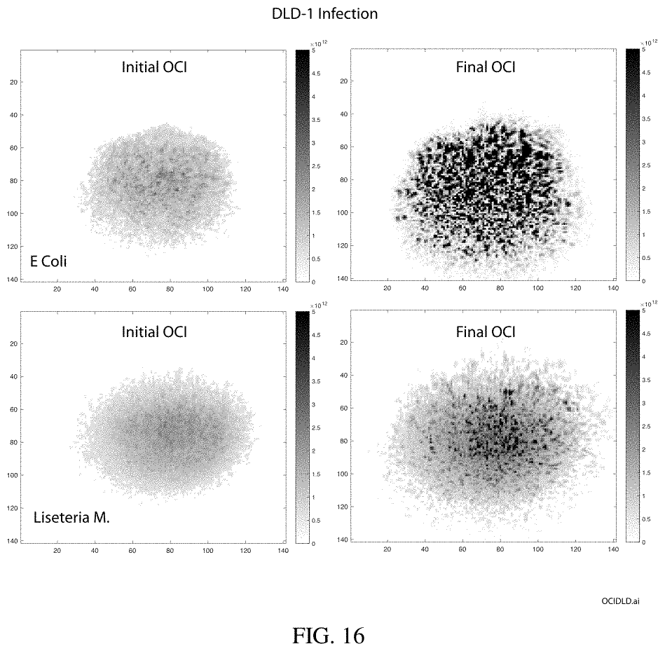

OCI images of DLD spheroids, before and after infection by E. coli and Listeria monocytogenes, are shown in FIG. 16. Infection is associated with an increase in backscatter brightness by several fold. The tissue also appears to expand, but this may be an artifact of the higher brightness affecting the masking threshold during image analysis. The higher brightness could be as simple as increased scattering from the bacteria themselves. It can also be a consequence of greater optical heterogeneity induced by the infection as the bacteria disrupt the functions and structures of the cells. The OCI images appear physically more heterogeneous after the infection. However, the fact that E. coli does not internalize inside the cells suggests that the increased brightness is directly caused by the increase in the number of bacteria scattering light. (It is important to keep in mind that the increased brightness can affect the calculation of the motility (NSD) and can induce a shift in the fluctuation spectra.)

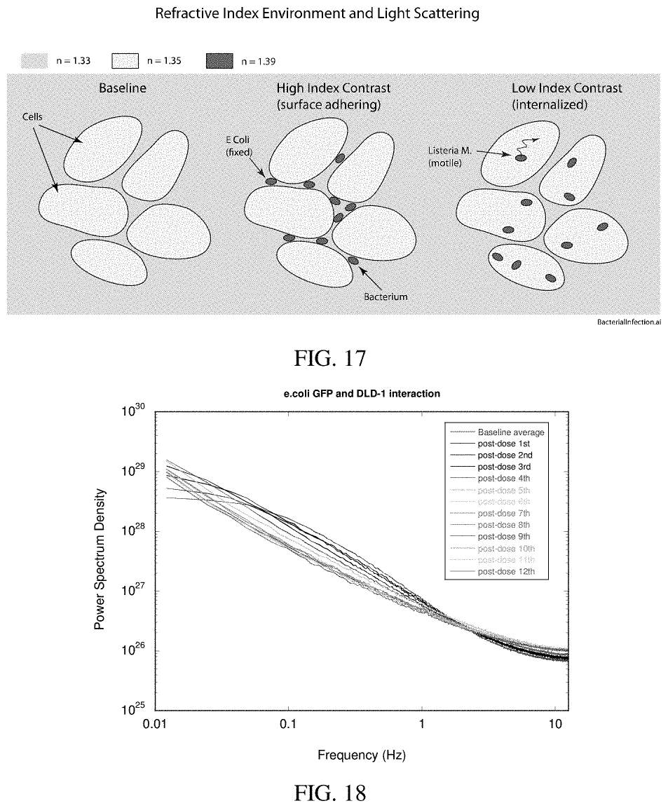

The large increase in integrated backscatter brightness for E. coli but not Listeria or Salmonella (see FIG. 15b) may relate the different ways that these bacteria interact with the host cells. For instance, E. coli adheres to the cell surface, while Salmonella and Listeria are both internalized, as shown in FIG. 17. The bacteria have high refractive index, which can be partially masked when they are surrounded by the high-index cytosol of the cells as opposed to the surrounding medium (refractive index similar to water). This can produce more scattering in the case of E. coli than for internalized bacteria and may serve as a marker for bacterial interaction and hence help provide a partial identification.

Tissue Dynamics Spectroscopy (TDS) of Tissue Infection

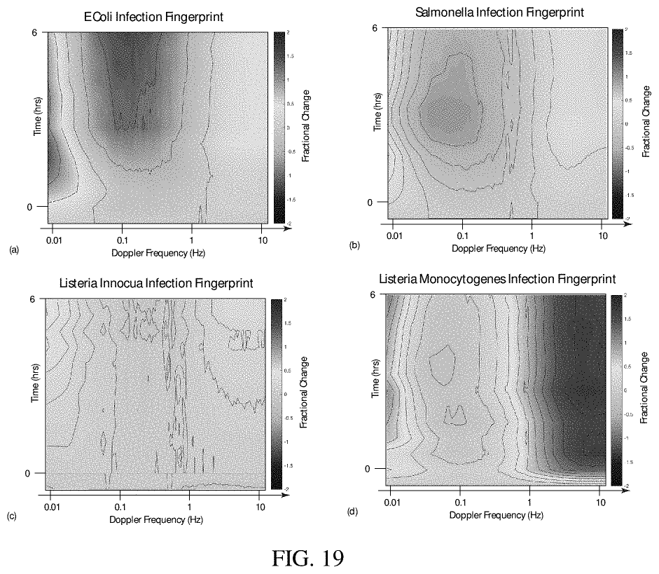

The hypothesis of intracellular motility decrease offset by increase in bacterial motility is supported by the tissue-response spectrograms that identify different spectral ranges that are affected by the bacterial infection. The spectra of DLD-1 under E. coli infection is shown in FIG. 17. The initial spectrum of the DLD-1 has a knee frequency feature at 0.1 Hz (corresponding to a root-mean squared intracellular speed of 30 nm/sec). When exposed to E. coli at a concentration of 10⁷ #/mL, the spectral shape is converted to a 1/f spectrum with strong suppression of the Doppler knee in the mid frequency range, accompanied by an increase in the low-frequency cell-shape band and a slight increase in the high-frequency Nyquist floor. The TDS spectrogram is shown in FIG. 18 showing the strong suppression of the Doppler edge.

TDS spectrograms of Salmonella, Listeria innocua and Listeria monocytogenes are shown in FIG. 19 compared to the spectrogram for E. coli. They all share similar frequency features because of the underlying use of the same DLD-1 cell line for each case. There is a characteristic frequency at 0.03 Hz that bounds the mid-frequency and low-frequency behaviors. There is another characteristic frequency at 1 Hz that separates the mid-frequency and high-frequency behaviors. The TDS spectrogram for Listeria monocytogenes shows by far the strongest effects that occur primarily at the high frequency range.

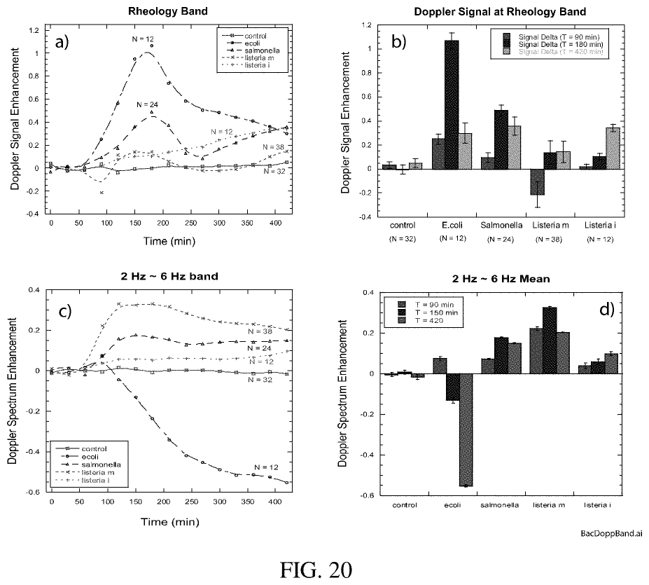

The time traces of selected spectral ranges are shown in FIG. 20 for the rheology band (10 mHz) and the organelle transport band (2 Hz-6 Hz) for an applied exposure of 10⁷ cfu per ml. The rheology band is associated with slow cellular shape changes and also with reduced speeds of mid-frequency processes related to membranes or larger organelles like the nucleus. At the high bacterial load of 10⁷ there is a strong non-monotonic time dependence of the spectral density of the rheology band as a maximum appears after approximately 2 hours and then the signal decreases. In the case of Salmonella and L. mono the secondary minimum gives way to a later increase, while for the non-pathogenic E. coli and L. innocua the secondary minimum does not occur. The organelle band presents a relatively “clean” signal, because the only intracellular constituents that contribute to this spectral range are the smaller organelles and vesicles. The spectral density of the organelle band is enhanced for all but E. coli infection. Organelle and vesicle transport are associated with active cellular responses to xenobiotic insults as well as with early-stage apoptosis. These frequencies are be associated with the motion of the bacteria themselves.

FIG. 20 provides time development of the relative spectral changes for a dose of 10⁷ cfu. The bacteria are added at 60 minutes followed by rapid changes in the spectral power. a) The low-frequency limit for the rheology band. All bacterial strains, except L. innocua, display increased activity in this band. E. coli displays the strongest increase with a maximum around 1 hour after infection. The two pathogenic strains, Salmonella and L. mono, show non-monotonic increases, with a decrease after the first maximum, followed by a long-term increase. Long-term increases for the rheology band have been associated with blebbing or the formation of apoptotic bodies associated with either uncontrolled or controlled cell death. b) Change in the Doppler rheology band for three selected times. c) Time dependence of the high-frequency band associated with organelle transport. Both pathogenic strains Salmonella and L. mono show strong initial increases within one hour with a slow decrease at longer times. The case for E. coli shows strong suppression consistent with an overall inhibition of Doppler activity. d) Change in the Doppler organelle transport band at three selected times.

Tissue Sentinel Response to Antibiotics

One of the most important applications of the tissue sentinels is to serve as a dynamic substrate for the study of antibiotic sensitivity and resistance. In previous studies of biodynamic imaging in drug development and personalized medicine the response of living biopsy samples, from patients enrolled in clinical trials exposed to standard-of-care chemotherapeutics, allowed the classification of patients into cohorts that are deemed sensitive or resistant to a given treatment (see Choi, H. G., Z. Li, H. Sun, D. Merrill, J. Turek, M. Childress and D. Nolte. “Biodynamic digital holography of chemo-resistance in a pre-clinical trial of canine B-cell lymphoma.” Biomedical Optics Express, 9(5): 2251-2265. (2018)). In the current microbiology context, the test samples become the immortalized tissue sentinels that perform as dynamic substrates on which to observe the dynamic effects of infection, but just as importantly to observe how the infection responds to antibiotic treatments. This embodiment makes it possible to identify biodynamic spectral signatures that correlate with efficacy of the antibiotic. The method is different than in Choi et al. because of the two-stage delivery of agents to the wells containing the sentinels. In the antibiotic assay, bacteria are delivered to a well containing a tissue sentinel, the infection is allowed to proceed for a selected time that is selected to allow a specified number of doublings. For instance, times allowing 3 or 6 or 12 doublings may be selected corresponding to about 1 or 2 or 4 hours of infection. Then the antibiotic is applied and the biodynamic spectra are acquired for a duration related to the infection time

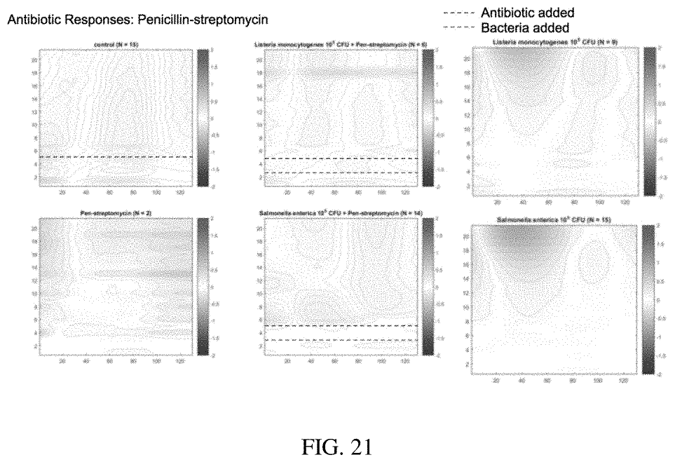

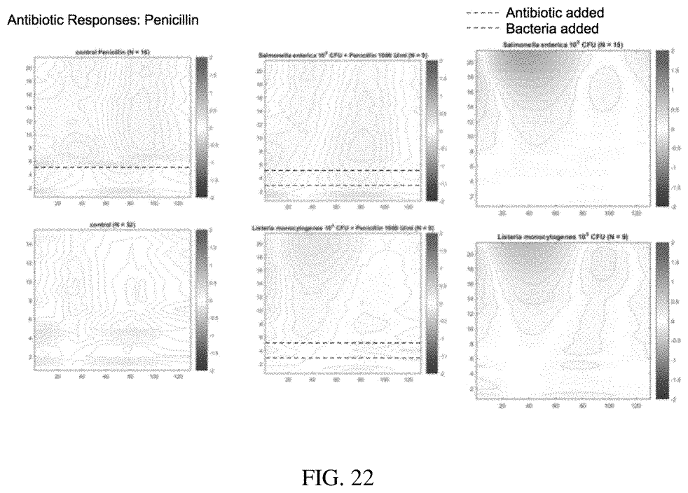

An example of an antibiotic treatment is shown in FIG. 21 and FIG. 22. In FIG. 21 a DLD-1 sentinel is used to study the effects of the antibiotic combination of penicillin and streptomycin on infections by Listeria and Salmonella. On the left of the figure are two negative controls. The upper control is simply the time progression of the spectral changes for normal DLD-1 spheroids. The lower control is the antibiotic applied to the tissue but without infection. The DLD tissues infected by the bacterial strains are shown on the right. For both Listeria and Salmonella there is a marked response to the infection that enhances low and high frequencies while suppressing the mid frequencies. In the two middle spectrograms, the tissues were infected by the bacteria, then the antibiotic penicillin plus streptomycin was applied about 1 hour later. For both Listeria and Salmonella, the antibiotic treatment prevented the onset of bacterial infection noted by the absence of the bacterial suppression (blue mid frequency) at late times.

Similar behavior is observed in FIG. 22 when only penicillin is applied without streptomycin. However, in the case of L. mono there is a weak infection signal that emerges about 8 hours after the antibiotic is applied indicating that this strain of L. mono is not completely inhibited by penicillin. Examples like this illustrate how this methodology would be used in an antibiotic sensitivity assay, in which delays in infection onset or complete absence of infection, as observed through the characteristic spectral changes associated with bacteria, determine the efficacy of the applied antibiotic.

Those skilled in the art will recognize that numerous modifications can be made to the specific implementations described above. The implementations should not be limited to the particular limitations described. Other implementations may be possible.

We claim:

1. A method for detection of pathogens in viable microorganisms, wherein the method comprises a) interacting living immortalized cells with viable microorganisms, and b) measuring changes in Doppler shift.

2. The method of claim 1, wherein the method comprises detecting pathogens through expressed metabolites from said viable microorganisms by measurements based on Doppler shift.

3. The method of claim 1, wherein the pathogens are represented in a pellet containing between 100 and 10,000 viable microorganisms.

4. The method of claim 3, wherein the pellets comprise toxins associated with living (viable) or dead bacterial cells.

5. The method of claim of claim 3, wherein the microorganisms may be either gram positive or gram negative bacteria.

6. The method of claim 3, wherein the viable microorganisms are concentrated and recovered from aqueous extracts of biological materials selected from the group consisting of food produce, fruit, meats, and dairy products.

7. The method of claim 1, wherein said viable microorganisms are obtained by a combination of microfiltration and/or ultrafiltration to concentrate viable microorganisms to achieve sufficient number of living bacteria needed to achieve detection of the presence of said microorganisms, wherein the number of the living bacteria is in the range of 1-10,000.

8. The method of claim 1, wherein said viable microorganisms comprises living and nonpathogenic bacteria.

9. The method of claim 1, wherein the detection of pathogens is achieved within 8 hours.

10. The method of claim 8, wherein interrogation of nonpathogenic cells is completed in 8 hours.

11. The method of claim 2, wherein expressed metabolites are obtained from viable microorganisms extracted from a food produce to achieve toxin concentrations needed to achieve detection as biomarkers associated with pathogens within 8 hours.

12. The method of claim 1, wherein the method comprises using living immortalized cells to detect presence of monocultures of viable microorganisms pathogens.

13. The method of claim 1, wherein said pathogens are detected by using a living tissue fragment of the living immortalized cells as a sensitive detector.

14. The method of claim 1, wherein the living immortalized cells are living immortalized cancer cells.

15. The method of claim 14, wherein the cancer cells are colon cancer cells.
